(19) United States
(12) Patent Application Publication
(10) Pub. No.: US 2008/0317832 A1
(43) Pub. Date: Dec. 25, 2008

(54) TRANSDERMAL DELIVERY SYSTEM COMPRISING GLYCOPYRROLATE TO TREAT SIALORRHEA

(75) Inventor: LARRY DILLAHA, Atlanta, GA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Sciele Pharma, Inc., Atlanta, GA (US)

(21) Appl. No.: 12/144,285

(22) Filed: Jun. 23, 2008

Related U.S. Application Data
(60) Provisional application No. 60/945,758, filed on Jun. 22, 2007.

(51) Int. Cl. A61K 31/4015; A61K 9/70; A61P 25/00

(52) U.S. Cl. 424/449; 514/424

(57) ABSTRACT

In one aspect, the invention includes a method for treating sialorrhea, comprising the steps of identifying a patient afflicted with sialorrhea and administering a therapeutically effective amount of glycopyrrolate to the patient using a transdermal route of administration. In another aspect, the invention is a transdermal drug delivery system for treating a patient exhibiting sialorrhea, including a transdermal patch, a therapeutically effective amount of glycopyrrolate contained in the transdermal patch to alleviate sialorrhea, and a pharmaceutically acceptable carrier. The transdermal patch can be a single layer drug-in-adhesive patch, a multi-layer drug-in-adhesive patch, a matrix patch, or a reservoir patch.

### CROSS-REFERENCE TO RELATED APPLICATIONS

This patent application claims the benefit of U.S. Provisional Patent Application No. 60/945,758, filed Jun. 22, 2007, which is incorporated by reference.

### TECHNICAL FIELD OF THE INVENTION

The present invention is directed to glycopyrrolate compositions and methods of administration to treat specific conditions. More specifically, the present invention is directed to treatment of conditions such as sialorrhea, hyperhidrosis, gustatory sweating, and Frey's syndrome with the transdermal administration of glycopyrrolate compositions.

### BACKGROUND

Glycopyrrolate, the active pharmaceutical ingredient in Robinul® tablets, Robinul® Forte tablets, and Robinul® injection, is a quaternary ammonium compound having the chemical name 3-[(cyclopentylhydroxyphenylacetyl)oxy]-1,1-dimethylpyrrolidinium bromide. Glycopyrrolate is an anticholinergic and antimuscarinic agent. Glycopyrrolate is indicated for use as a preoperative antimuscarinic to reduce salivary, tracheobronchial, and pharyngeal secretions. See Physicians' Desk Reference (57th ed., Medical Economics Co., 2003). Glycopyrrolate also is used to treat the symptoms of some neurological disorders. In particular, glycopyrrolate can be used to reduce excessive saliva that can pool in the mouth or leak out. This condition is known as sialorrhea (persistent or excessive drooling).

Persistent or excessive drooling beyond the age of three years is considered abnormal. Such drooling may be found in individuals with neurological dysfunction or motor deficits (e.g., cerebral palsy, peripheral neuromuscular disease, facial paralysis, and mental retardation) and other conditions such as esophageal cancer. Drooling causes impairment of speech, feeding and swallowing problems, upper respiratory congestion, and choking upon aspiration. Control of drooling is important in preventing choking and gagging in persons with posterior drooling.

Sialorrhea can cause a range of physical and psychosocial complications, including perioral chapping, dehydration, odor, and social stigmatization that can be devastating for patients and their families. Current recommendations for treating sialorrhea include a clinical team of primary health care providers, speech pathologists, occupational therapists, dentists, orthodontists, neurologists, and otolaryngologists. Treatment options range from conservative (i.e., observation, postural changes, and biofeedback) to more aggressive measures such as radiation, surgical intervention, and medication.

The ingestion of anticholinergic medications, such as glycopyrrolate, is effective in reducing drooling but it may be difficult to administer these medications to patients who have trouble swallowing. Accordingly, there is a need in the art for an easily administrable preparation comprising glycopyrrolate for patients (e.g., aged, disabled, or pediatric patients) who have difficulty swallowing.

There is a need in the art for a preparation of glycopyrrolate that specifically addresses the problem of sialorrhea while providing a convenient and effective administration route for patients or individuals who may not easily benefit from the drug otherwise.

### SUMMARY OF THE INVENTION

The present invention comprises systems and methods for the treatment of sialorrhea. More specifically, the systems and methods comprise noninvasive, transdermal administration of glycopyrrolate to treat sialorrhea.

Transdermal drug delivery (TDD) offers several advantages over traditional delivery methods including injections and oral delivery. When compared to oral delivery, TDD avoids gastrointestinal drug metabolism, reduces first pass liver metabolism effects, and provides sustained release of glycopyrrolate compositions. In actuality, transdermal delivery is transport of glycopyrrolate compositions across the epidermis where the glycopyrrolate compositions are absorbed by the blood capillaries. When compared to injections, TDD eliminates the associated pain and the possibility of infection.

In one aspect, the invention is a method for treating sialorrhea, comprising the steps of identifying a patient afflicted with sialorrhea and administering a therapeutically effective amount of glycopyrrolate to the patient using a transdermal route of administration. The therapeutically effective amount of glycopyrrolate can be from about 0.0001 mg/kg/day to 300 mg/kg/day. Alternatively, the therapeutically effective amount of glycopyrrolate can be from about 0.0005 mg/kg/day to about 50 mg/kg/day. In addition, the therapeutically effective amount of glycopyrrolate can be from about 0.001 mg/kg/day to about 10 mg/kg/day.

In one embodiment, the method of the present invention can be used to alleviate sialorrhea in a patient suffering from a neurological dysfunction. For example, the neurological dysfunction may be Parkinson's disease, stroke, cerebral palsy, amyotrophic lateral sclerosis, or mental retardation. By way of further example, the method of the present invention may benefit a patient suffering from facial paralysis or cancer about the face, neck, or esophagus.

The method of the present invention can include several transdermal routes of administration, including a single layer drug-in-adhesive patch, a multi-layer drug-in-adhesive patch, a matrix patch, or a reservoir patch. Typically, transdermal patches incorporate at least one adhesive to adhere the patch to the patient. Adhesives can include acrylics, vinyl acetates, natural and synthetic rubbers, ethylene-vinyl acetate copolymers, polysiloxanes, polyacrylates, polyurethanes, plasticized polyether block amide copolymers, plasticized styrene-rubber block copolymers, and mixtures thereof.

In another embodiment, the method of the present invention includes at least one skin penetration enhancer (i.e., enhancer) to enhance penetration of transdermally-administered glycopyrrolate. Skin penetration enhancers can include, for example, fatty acids or salts thereof, fatty alcohols, branched aliphatic alcohols, fatty acid alkyl esters, fatty acid monoesters of sorbitol and glycerol, fatty acid esters with glycolic acid and lactylic acid and salts thereof, fatty acid amides, alkylpyrrolidones, or mixtures thereof.

In another aspect, the invention is a transdermal drug delivery system for treating a patient exhibiting sialorrhea, including a transdermal patch, a therapeutically effective amount of glycopyrrolate contained in the transdermal patch to alleviate sialorrhea, and a pharmaceutically acceptable carrier. As mentioned above, the transdermal patch can be a single layer drug-in-adhesive patch, a multi-layer drug-in-adhesive patch, a matrix patch, or a reservoir patch.

The transdermal drug delivery system of the present invention includes the aforementioned adhesives to adhere the transdermal patch to the patient as well as skin penetration enhancers to facilitate the penetration of glycopyrrolate through the patient's skin. Further, the therapeutically effective amounts of glycopyrrolate (i.e., from about 0.0001 mg/kg/day to about 300 mg/kg/day, or from about 0.0005 mg/kg/day to about 50 mg/kg/day, or from about 0.001 mg/kg/day to about 10 mg/kg/day) apply to the transdermal drug delivery system of the present invention.

In one embodiment, the transdermal drug delivery system of the present invention includes a viscous material suitable for inclusion in a reservoir patch as the pharmaceutically acceptable carrier.

In another embodiment, the transdermal drug delivery system of the present invention includes a biocompatible polymer suitable for inclusion in a matrix patch as the pharmaceutically acceptable carrier.

In yet another embodiment, the transdermal drug delivery system of the present invention further includes a pharmaceutically acceptable counter ion. Pharmaceutically acceptable counter ions include chloride, bromide, iodide, acetate, 2-ethylhexanoate, sulfate, phosphate, arylsulfonates, cyclohexylsulfamate, benzoate, saccharinate, or a mixture thereof.

### DETAILED DESCRIPTION OF THE INVENTION

It will be understood that the terminology herein is used for the purpose of describing particular embodiments only and is not intended to be limiting. The scope of the present invention will be limited only by the appended claims.

It must be noted that, as used in this specification and the appended claims, the singular forms “a,” “an,” and “the” include plural referents unless the context clearly dictates otherwise. For example, reference to a multilayer patch containing “a glycopyrrolate composition” can include a mixture of such compositions and reference to “an adhesive” can include reference to one or more of such adhesives.

As used herein, “pharmaceutically effective amount” or “effective amount” means an amount of a glycopyrrolate composition that is sufficient to provide a selected effect and performance (i.e., alleviate sialorrhea) at a reasonable benefit/risk ratio attending any medical treatment. An effective amount of a skin penetration enhancer as used herein means an amount selected so as to provide the selected increase in permeability and the desired depth of penetration, rate of administration, and amount of drug delivered.

As used herein, “skin penetration enhancer,” “skin permeation enhancer,” and the like shall be inclusive of all enhancers that increase the flux of a permeant, drug, or other molecule across the skin or mucosa and is limited only by functionality. In other words, all cell envelope disordering compounds, solvents, steroidal detergents, bile salts, chelators, surfactants, non-surfactants, fatty acids, and any other chemical enhancement agents are intended to be included.

As used herein, “adhesive,” “adhesive polymer,” “mucoadhesive,” or such similar terms refers to hydrophilic polymers, natural or synthetic, which, by the hydrophilic designation, can be either water soluble or swellable and which are compatible with the enhancers and glycopyrrolate compositions. The adhesives may even function to adhere the dosage forms to the mucous tissues of the oral cavity, such as the gingiva. Such adhesives are inclusive of hydroxypropyl cellulose, hydroxypropyl methylcellulose, hydroxy ethylcellulose, ethylcellulose, carboxymethyl cellulose, dextran, guar gum, polyvinyl pyrrolidone, pectins, starches, gelatin, casein, acrylic acid polymers, polymers of acrylic acid esters, acrylic acid copolymers, vinyl polymers, vinyl copolymers, polymers of vinyl alcohols, alkoxy polymers, polyethylene oxide polymers, polyethers, and mixtures thereof and the like.

The terms, “system,” “drug delivery system,” “transmucosal delivery system,” or the like mean a unit dosage form of a drug composition, preferably glycopyrrolate compositions, including carriers, enhancers, and other components, in which the glycopyrrolate composition is contained in or accompanied by means for maintaining the drug composition in a drug transferring relationship or providing the glycopyrrolate compositions to the desired site in the body. For example, the means used can be a device such as a matrix patch or liquid reservoir patch as hereinafter described.

The method of application of the present invention can vary within limits, but necessarily involves providing the selected glycopyrrolate compositions to the patient such that drug delivery is initiated and continues for a period of time sufficient to provide the selected pharmacological or biological response, i.e., alleviation of sialorrhea.

The systems and methods of the present invention comprise glycopyrrolate, or alternatively can contain glycopyrronium bromide. In addition, the compositions of the present invention comprise delivery vehicles or permeation enhancers known to those skilled in the art.

The present invention comprises systems and methods for the treatment of sialorrhea. More specifically, the systems and methods comprise noninvasive, transdermal administration of glycopyrrolate to treat sialorrhea.

In one aspect, the invention is a method for treating sialorrhea, comprising the steps of identifying a patient afflicted with sialorrhea and administering a therapeutically effective amount of glycopyrrolate to the patient using a transdermal route of administration. The therapeutically effective amount of glycopyrrolate can be from about 0.0001 mg/kg/day to 300 mg/kg/day (e.g., about 0.0002 mg/kg/day, about 0.0005 mg/kg/day, about 0.0007 mg/kg/day, about 0.001 mg/kg/day, about 0.01 mg/kg/day, about 0.1 mg/kg/day, about 1 mg/kg/day, about 10 mg/kg/day, about 100 mg/kg/day, about 200 mg/kg/day, and ranges thereof). Alternatively, the therapeutically effective amount of glycopyrrolate can be from about 0.0005 mg/kg/day to about 50 mg/kg/day (e.g., about 0.001 mg/kg/day, about 0.005 mg/kg/day, about 0.01 mg/kg/day, about 0.05 mg/kg/day, 0.06 mg/kg/day, about 0.1 mg/kg/day, about 0.5 mg/kg/day, about 1 mg/kg/day, about 5 mg/kg/day, about 10 mg/kg/day, about 20 mg/kg/day, about 30 mg/kg/day, about 40 mg/kg/day, or ranges thereof). In addition, the therapeutically effective amount of glycopyrrolate can be from about 0.001 mg/kg/day to about 10 mg/kg/day (e.g., about 0.005 mg/kg/day, about 0.01 mg/kg/day, about 0.05 mg/kg/day, about 0.1 mg/kg/day, about 0.15 mg/kg/day, about 0.18 mg/kg/day, about 0.2 mg/kg/day, about 0.24 mg/kg/day, about 0.3 mg/kg/day, about 0.5 mg/kg/day, about 0.7 mg/kg/day, about 1 mg/kg/day, about 2 mg/kg/day, about 3 mg/kg/day, about 4 mg/kg/day, about 5 mg/kg/day, about 6 mg/kg/day, about 7 mg/kg/day, about 8 mg/kg/day, about 9 mg/kg/day, and ranges thereof). In particular, the therapeutically effective amount of glycopyrrolate to be delivered via transdermal administration can be about 3 mg/day, about 6 mg/day, or about 9 mg/day. The therapeutically effective amount may need to be titrated to each individual patient depending on the severity of the sialorrhea.

In one embodiment, the method of the present invention can be used to alleviate sialorrhea in a patient suffering from a neurological dysfunction. For example, the neurological dysfunction can be Parkinson's disease, stroke, cerebral palsy, amyotrophic lateral sclerosis, or mental retardation. By way of further example, the method of the present invention may benefit a patient suffering from facial paralysis or cancer about the face, neck or esophagus.

The method of the present invention can include several transdermal routes of administration, including a single layer drug-in-adhesive patch, a multi-layer drug-in-adhesive patch, a matrix patch, or a reservoir patch (i.e., a liquid reservoir system, or LRS). Typically, transdermal patches incorporate at least one adhesive to adhere the patch to the patient. Adhesives can include acrylics, vinyl acetates, natural and synthetic rubbers, ethylene-vinyl acetate copolymers, polysiloxanes, polyacrylates, polyurethanes, plasticized polyether block amide copolymers, plasticized styrene-rubber block copolymers, and mixtures thereof.

In another embodiment, the method of the present invention includes at least one skin penetration enhancer to enhance penetration of transdermally-administered glycopyrrolate. Skin penetration enhancers can include, for example, fatty acids or salts thereof, fatty alcohols, branched aliphatic alcohols, fatty acid alkyl esters, fatty acid monoesters of sorbitol and glycerol, fatty acid esters with glycolic acid and lactylic acid and salts thereof, fatty acid amides, alkylpyrrolidones, or mixtures thereof.

In another aspect, the invention is a transdermal drug delivery system for treating a patient exhibiting sialorrhea, including a transdermal patch, a therapeutically effective amount of glycopyrrolate contained in the transdermal patch to alleviate sialorrhea, and a pharmaceutically acceptable carrier. As mentioned above, the transdermal patch can be a single layer drug-in-adhesive patch, a multi-layer drug-in-adhesive patch, a matrix patch, or a reservoir patch (LRS).

Generally speaking, the various types of patches are thus described. The single layer drug-in-adhesive patch includes an adhesive layer that also contains the drug. In this type of patch, the adhesive layer serves to adhere the various layers together and can also adhere the entire system to the skin. Furthermore, the adhesive layer can also be responsible for the releasing the drug. The adhesive layer can be surrounded by a temporary liner and a backing.

The multi-layer drug-in adhesive patch is similar to the single-layer system in that multiple adhesive layers are responsible for releasing the drug. The multi-layer system is different in that it can add another layer of drug-in-adhesive, usually separated by a membrane (but not in all cases). This patch can have a temporary liner-layer and a permanent backing.

The matrix patch typically has a drug layer of a semisolid matrix containing a drug solution or suspension. The adhesive layer in this patch surrounds the drug layer partially overlaying it.

The reservoir transdermal patch typically has a separate drug layer. The drug layer is a liquid or gel compartment containing a drug solution or suspension separated by the adhesive layer. This patch is also backed by a backing layer.

One embodiment of the transdermal drug delivery system of the present invention can include structural components. For example, in the case of an adhesive matrix patch, a distal backing is laminated to the polymer layer. Such a distal backing defines the side of the matrix patch that faces the environment, i.e., distal to the skin or mucosa. The backing layer functions to protect the matrix polymer layer and drug/enhancer composition and to provide an impenetrable layer that prevents loss of drug to the environment. Thus, the material chosen for the backing should be compatible with the polymer layer, drug, and enhancer, and should be minimally permeable to any components of the matrix patch. Advantageously, the backing can be opaque to protect components of the matrix patch from degradation from exposure to ultraviolet light. Furthermore, the backing should be capable of binding to and supporting the polymer layer, yet should be pliable enough to accommodate the movements of a person using the matrix patch.

Suitable materials for the backing include, but are not limited to: metal foils, metalized polyfoils, composite foils or films containing polyester such as polyester terephthalate, polyester or aluminized polyester, polytetrafluoroethylene, polyether block amide copolymers, polyethylene methyl methacrylate block copolymers, polyurethanes, polyvinylidene chloride, nylon, silicone elastomers, rubber-based polyisobutylene, styrene, styrene-butadiene and styrene-isoprene copolymers, polyethylene, and polypropylene. In one aspect of the invention, the backing layer can have a thickness of from about 0.0005 inch to about 0.01 inch (e.g., from about 0.0007 inch to about 0.007 inch, from about 0.0009 inch to about 0.005 inch, or from about 0.001 inch to about 0.0003 inch).

Further, a release liner can be temporarily provided upon the proximal side (i.e., side to adhere to the skin) of the adhesive layer. Such a liner provides many of the same functions as the backing layer, prior to adhesion of the patch to the skin. In use, the release liner is peeled from the adhesive layer just prior to application and discarded. The release liner can be made of the same materials as the backing layer, or other suitable films coated with an appropriate release surface.

The transdermal drug delivery system of the present invention includes the aforementioned adhesives to adhere the transdermal patch to the patient as well as skin penetration enhancers to facilitate the penetration of glycopyrrolate through the patient's skin. In one embodiment of the present invention, pressure-sensitive adhesives are suitable for long-term (e.g., greater than 1 day, such as about 2 days, about 3 days, about 4 days, about 5 days, about 6 days, about 1 week, about 2 weeks, about 3 weeks, or about 4 weeks) contact with the skin. In another embodiment, the pressure-sensitive adhesive of the carrier is suitable for a short-term administration (e.g., for a few minutes to a few hours, but less than or equal to 1 day, such as about 1 minute, about 5 minutes, about 10 minutes, about 30 minutes, about 45 minutes, about 60 minutes, about 90 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, or about 16 hours). Such adhesives must be physically and chemically compatible with the drug and skin penetration enhancer, and with any carriers and/or vehicles or other additives incorporated into the drug/enhancer composition.

In yet another embodiment of the present invention, the adhesives of the pharmaceutically acceptable carrier include without limitation, acrylic adhesives including cross-linked and uncross-linked acrylic copolymers; vinyl acetate adhesives; natural and synthetic rubbers including polyisobutylenes, neoprenes, polybutadienes, and polyisoprenes; ethylenevinylacetate copolymers; polysiloxanes; polyacrylates; polyurethanes; plasticized weight polyether block amide copolymers, and plasticized styrene-rubber block copolymers or mixtures thereof.

The pharmaceutically acceptable carrier of the present invention can be made of a wide variety of materials known to those skilled in the art of transdermal drug delivery. In one embodiment of the present invention the carrier can be a biocompatible polymer. In another embodiment, the carrier can be one of the aforementioned adhesives. For example, the carrier in an adhesive matrix patch can be a biocompatible adhesive polymer. In the case of an LRS patch, the carrier forms a gel, or other viscous form suitable for use in an LRS patch.

In addition to containing glycopyrrolate, the pharmaceutically acceptable carrier can comprise a number of other additives, such as diluents, excipients, emollients, plasticizers, skin irritation reducing agents, or a mixture thereof. For example, suitable diluents can include mineral oil, low molecular weight polymers, plasticizers, and the like. Many transdermal drug delivery formulations have a tendency to cause skin irritation after prolonged exposure to the skin, thus addition of a skin irritation reducing agent aids in achieving a composition that is better tolerated by the skin. In one embodiment, the skin irritation reducing agent can be glycerin, as disclosed in U.S. Pat. No. 4,855,294.

The therapeutically effective amounts of glycopyrrolate (i.e., from about 0.0001 mg/kg/day to about 300 mg/kg/day, or from about 0.0005 mg/kg/day to about 50 mg/kg/day, or from about 0.001 mg/kg/day to about 10 mg/kg/day) apply to the transdermal drug delivery system of the present invention. The transdermal drug delivery system of the present invention can include transdermal patches having different amounts of glycopyrrolate to allow for drug dosage titration depending on the individual needs of the patient.

In yet another embodiment, the transdermal drug delivery system of the present invention further includes a pharmaceutically acceptable counter ion. A counter ion is an oppositely charged ion that accompanies an ionic species in order to maintain a balanced charge. Pharmaceutically acceptable counter ions include chloride, bromide, iodide, acetate, 2-ethylhexanoate, sulfate, phosphate, arylsulfonates, cyclohexylsulfamate, benzoate, saccharinate, or a mixture thereof.

In yet another embodiment, the transdermal drug delivery system of the present invention includes at least one skin penetration enhancer. Skin penetration enhancers can be comprised of two primary categories of components. One category is cell-envelope disordering compounds. The second category can be solvents or binary systems containing both cell-envelope disordering compounds and solvents. Other categories of skin penetration enhancers are also known (e.g., steroidal detergents, bile salts, chelators, surfactants, non-surfactants, and fatty acids).

Cell envelope disordering compounds are known as being useful in topical pharmaceutical preparations and aid in drug delivery through the skin or mucosa. These compounds assist in dermal penetration by disordering the lipid structure of the stratum corneum cell-envelopes. A list of such compounds appears in, for example, U.S. Pat. No. 5,780,050, which is incorporated herein by reference.

Suitable solvents include water; diols, such as propylene glycol and glycerol; mono-alcohols, such as ethanol, propanol, and higher alcohols; DMSO; dimethylformamide; N,N-dimethylacetamide; 2-pyrrolidone; N-(2-hydroxyethyl)pyrrolidone, N-methylpyrrolidone, 1-dodecylazacycloheptan-2-one, and other n-substituted alkyl-azacycloalkyl-2-ones (azones) and the like.

As used herein, “bile salts” means steroidal detergents that are the natural or synthetic salts of cholanic acid, such as the salts of cholic and deoxycholic acid or combinations of such salts, including the unionized acid form. Bile salt analogs having the same physical characteristics and that also function as permeation enhancers are also included in this definition.

More specifically, the transdermal drug delivery system of the present invention can contain, without limitation, at least one skin penetration enhancer including oleic acid; lauric acid; oleyl alcohol; lauryl alcohol; 2-butyl-octanol; 2-hexyl decanol; 2-octyl-decanol; 2-hexyldodecanol; 2-octyl-dodecanol; 2-decyl-tetradecanol; 2-tetradecyl-octadecanol; methyl and ethyl laurate; sorbitan monooleate and monolaurate; glycerol monooleate and monolaurate; lauric, myristic, capric, stearic, and oleic diethanolamide; lauric, myristic, capric, stearic, and oleic monoethanolamide; lauric, myristic, capric, stearic, and oleic monoisopropanolamide; caproyl, lauroyl and stearoyl lactylic acid and their salts; caproyl, lauroyl and stearoyl glycolic acid and their salts; N-n-octyl and N-n-dodecyl pyrrolidone.

The following example further illustrates the invention but, of course, should not be construed as in any way limiting its scope.

### EXAMPLE

This example demonstrates procedures for determining a therapeutically effective amount of glycopyrrolate in treating sialorrhea. The following study was designed for oral administration of liquid glycopyrrolate, however, the study is easily modified in the spirit and scope of the invention to assess transdermal administration of glycopyrrolate (e.g., by a transdermal patch).

I. Pre-Study Evaluation, Screening, and Baseline are as Described Below.

Prospective patients are screened for the study up to three weeks prior to dosing. Patients receiving anti-sialogenic compounds or other medications with anticholinergic or cholinergic activity must undergo a washout phase prior to the study baseline data collection phase which, for the study, begins on Day −8 before randomization.

At screening, the patient's eligibility for the study is assessed utilizing the following criteria: demographic data; complete medical history; complete physical examination; weight, height, percentile for age; blood pressure, heart rate; temperature (oral temperature is preferred, as feasible); resting 12-lead electrocardiogram (ECG); and laboratory tests.

Laboratory tests include hematology, such as hemoglobin, hematocrit, red blood cells, platelets, white blood cells, and differential white blood cell count (i.e., neutrophils, basophils, eosinophils, lymphocytes, monocytes); blood chemistry, such as creatinine, blood urea nitrogen, sodium, potassium, chloride, bicarbonate, glucose, alkaline phosphatase, alanine aminotransferase, aspartate aminotransferase, gamma-glutamyl transferase, total bilirubin, calcium, phosphorus, uric acid, cholesterol, total protein, and albumin; urinalysis, such as dipstick (leukocytes, protein, blood, glucose, ketones), and, if abnormal, microscopic sediment examination (erythrocytes, leukocytes, bacteria, casts, epithelial cells); thyroid stimulating hormone and free thyroxine (in nonverbal, non-mobile patients) to screen for hyperthyroidism which can exist undetected in nonverbal, non-mobile patients; and urine or blood pregnancy test (if applicable).

Additionally, at screening (Days −21 to −9), each patient must have profuse, severe, drooling to the extent that, in the absence of treatment, clothing becomes damp most days (approximately 5-7 days per week). If, in the absence of treatment, clothing, including bibs, shirts, or other clothing such as headbands used to catch drooling, is changed during the day because of dampness due to drooling, this is considered profuse, severe drooling for that day.

After screening and before the baseline period, parents/caregivers are instructed on how to use the modified Teacher's Drooling Scale (as described in Table 1) and the modified Behavioral and Medical Rating Scale (mBMRS) as described below.

TABLE 1
9 Point Modified Teacher's Drooling Scale (mTDS)

| SCORE | DESCRIPTION |
|---|---|
| 1 | Dry: never drools |
| 2 | Mild: only the lips are wet; occasionally¹ |
| 3 | Mild: only the lips are wet; frequently² |
| 4 | Moderate: wet on lips and chin; occasionally |
| 5 | Moderate: wet on lips and chin; frequently |
| 6 | Severe: drools to the extent that clothing becomes damp; |
| | occasionally |
| 7 | Severe: drools to the extent that clothing becomes damp; |
| | frequently |
| 8 | Profuse: clothing, hands, tray, and objects become wet; |
| | occasionally |
| 9 | Profuse: clothing, hands, tray, and objects become wet; |
| | frequently |

¹Occasionally refers to less than half of the time
²Frequently refers to more than half of the time

II. The Dose Titration Period (Days 1-28) is as Described Below.

After screening and washout (if applicable), but before randomization, baseline salivary assessments using the modified Teacher's Drooling Scale 9-point scale are made on two days of the parent/caregiver's choice within a nine-day period (Day −8 to Day 0) by the parent/caregiver at the following times during the day: 7-8 AM, 9-10 AM, 3-4 PM and at bedtime, approximately 9-10 PM. These determinations are used as baseline values for the primary efficacy measure.

The mBMRS is an instrument used to assess medication-associated symptoms. It is used by parent/caregivers during the baseline period and 2-3 times weekly after randomization throughout the study. During the baseline period (Days −8 to 0), the mBMRS is administered twice: once each on two separate non-consecutive days of the parent/caregiver's choice. Each mBMRS assessment is completed for the overall day on which the assessment is made. To accomplish these mBMRS assessments and also the modified Teacher's Drooling Scale assessments at baseline and after randomization, the patient's diary is dispensed to the parent/caregiver during screening (Days −21 to −9) and at that time the parent/caregiver is instructed regarding how to use the diary and how to use the modified Teacher's Drooling Scale and mBMRS scale.

After all inclusion/exclusion criteria have been met and baseline assessments have been obtained, the patient is randomized at Visit 3 (Day +1) to one of the two treatment groups (glycopyrrolate liquid or placebo). Randomization can be performed after confirming all eligibility criteria have been met, the washout period for prohibited treatments, if applicable, has been completed, and baseline period assessments have been properly completed.

Study medication is to be given so as not to exceed 9 mg daily, typically titrated over the course of 24 hours or more.

During the first four weeks after randomization and by Visit 5 (Day 28), Dose-levels are titrated to optimal tolerated response beginning at 0.02 mg/kg (Dose-level 1 of the Dose Titration Schedule (see Table 2) The initial Dose-level is assigned during the randomization visit (Visit 3, Day +1) and the parent/caregiver is instructed how to measure this Dose-level.

Patients are assessed every five to seven days by the investigator during the four-week dose titration phase until optimal dose has been achieved. Changes in dose are based on the titration schedule using the Dose-levels provided. An optimal dose should be attained by Week 4 (Visit 5, Day 28).

The initial starting dose for all patients is Dose Level 1. Then, every 5-7 days, patients are titrated up one Dose Level only until the optimal dose is attained (i.e., the desired reduction in drooling is reached), undesirable side effects become limiting, or the highest dose in the titration schedule is reached, whichever comes first. No patient is to be dosed higher than 3 mg (15 mL) three times daily or Dose Level 5 three times daily, whichever is the lesser dose for the patient's weight category.

If side effects become intolerable, the parent/caregiver is instructed to reduce the dosage to the previous Dose-level in the Dose Titration Schedule and continue using that Dose-level for the remainder of the study, or until side effects require another reduction to the next-lowest Dose-level. Parents/caregivers also can reduce or stop doses of their own volition.

TABLE 2
Dose Titration Schedule

| Weight | | Dose Level 1 | | Dose Level 2 | | Dose Level 3 | | Dose Level 4 | | Dose Level 5 | |
|---|---|---|---|---|---|---|---|---|---|---|---|
| kg | lb | (~0.02 mg/kg) | | (~0.04 mg/kg) | | (~0.06 mg/kg) | | (~0.08 mg/kg) | | (~0.1 mg/kg) | |
| 13-17 | 27-38 | 0.3 mg* | 1.5 ml | 0.6 mg | 3 ml | 0.9 mg | 4.5 ml | 1.2 mg | 6 ml | 1.5 mg | 7.5 ml |
| 18-22 | 39-49 | 0.4 mg | 2 ml | 0.8 mg | 4 ml | 1.2 mg | 6 ml | 1.6 mg | 8 ml | 2.0 mg | 10 ml |
| 23-27 | 50-60 | 0.5 mg | 2.5 ml | 1.0 mg | 5 ml | 1.5 mg | 7.5 ml | 2.0 mg | 10 ml | 2.5 mg | 12.5 ml |
| 28-32 | 61-71 | 0.6 mg | 3 ml | 1.2 mg | 6 ml | 1.8 mg | 9 ml | 2.4 mg | 12 ml | 3.0 mg | 15 ml |
| 33-37 | 72-82 | 0.7 mg | 3.5 ml | 1.4 mg | 7 ml | 2.1 mg | 10.5 ml | 2.8 mg | 14 ml | 3.0 mg | 15 ml |
| 38-42 | 83-93 | 0.8 mg | 4 ml | 1.6 mg | 8 ml | 2.4 mg | 12 ml | 3.0 mg | 15 ml | 3.0 mg | 15 ml |
| 43-47 | 94-104 | 0.9 mg | 4.5 ml | 1.8 mg | 9 ml | 2.7 mg | 13.5 ml | 3.0 mg | 15 ml | 3.0 mg | 15 ml |
| ≧48 | ≧105 | 1.0 mg | 5 ml | 2.0 mg | 10 ml | 3.0 mg | 15 ml | 3.0 mg | 15 ml | 3.0 mg | 15 ml |

*Glycopyrrolate Liquid (1 mg/5 mL); described doses are to be given three times daily.

During the dose titration phase, efficacy measures are assessed as discussed below on study Days 14±3 and 28±3 by the parent/caregiver. The mBMRS is administered by the parent/caregiver three times weekly, every two to three days, during the overall eight-week trial. The investigator also administers the mBMRS as a scripted verbal questionnaire at Visits 4 and 5. The investigator continues to administer the mBMRS as a scripted verbal questionnaire on Visits 6 and 7. Adverse events and concomitant medications are recorded.

III. The Post-Titration Study Period (Days 29-56) is as Described Below.

After the optimal Dose-level has been achieved (at or before Visit 5, Day 28), patients continue to receive the blinded study medication for a total of eight weeks (56 days total).

During the post-titration study, the following procedures are performed:

Efficacy measures as discussed below are assessed on study Days 42±3, and 56±3 (weeks six and eight) by the parent/caregiver and the physician (Day 56±3 only).

Adverse events and concomitant medications are recorded. The mBMRS continues to be used by the parent/caregiver every two to three days throughout the study as well as by the investigator (as a scripted verbal questionnaire) on Visits 4, 5, 6 and 7. The investigator indicates whether adverse events or serious adverse events were identified by the parent/caregiver's use of the mBMRS, which permits data analysis to distinguish between adverse events identified by mBMRS and adverse events not identified by mBMRS. A resting 12-lead ECG is evaluated on Day 56 (Week 8) or at the Dropout visit. Laboratory tests as described above are evaluated on Day 56 (Week 8) or at the Dropout visit.

Efficacy assessments are performed throughout the study. Modified 9-point Teacher's Drooling Scale assessments are performed by the parent/caregiver at baseline (on two separate non-school days of the parent/caregiver's choice within the nine-day period of Day −8 to Day 0, before randomization) and on Days 14±3, 28±3, 42±3 and 56±3, (2, 4, 6, and 8 weeks) after randomization. On each of these non-school days, four modified Teacher's Drooling Scale assessments are obtained by the parent/caregiver who is with the patient all day at the following times: pre-dose, in the morning (before 7-8 AM dose), two hours post-dose (approximately 9-10 AM), two hours post mid-day dose (approximately 3-4 PM), and two hours after the third dose, at bedtime (approximately 9-10 PM) or just prior to retiring to bed, (but not earlier than one hour post-dose).

Throughout the study, each modified Teacher's Drooling Scale assessment by a parent/caregiver covers a 30-60 minute time period to evaluate both severity and frequency of drooling. When the study medication is initiated, for each subsequent dose change, and for each dose administered on days when the modified Teacher's Drooling Scale is administered, the parent/caregiver records the dose amount (mg), date, and time of the dose. For missed doses, the parent/caregiver notes the missed dose amount (mg) and date and time for each missed dose of study medication. The time(s) of dosing at school, if applicable, is provided by teachers to parents/caregivers. This permits documentation of the dose titration process and maintenance dosage throughout the course of the study for each patient.

If feasible, if a decision is made that a patient is to drop from the study, the set of modified Teacher's Drooling Scale assessments described above is completed by the parent/caregiver on the last day of complete dosing. Additional dosing may not be possible if the patient drops from the study due to an adverse event.

Additional efficacy assessments include parent/caregiver's global assessments at Week 8 (or earlier if the patient discontinues participation in the trial); physician's global assessments at the last visit, Week 8 (or earlier if the patient discontinues participation in the trial), and patient's global assessments at the last visit, Week 8 (or earlier if the patient discontinues participation in the trial), only for patients who are deemed cognitively capable by the investigator. Very young patients, 3 to 8 year olds for example, complete a patient's global assessment only if the investigator determines that they are cognitively capable of doing so. A subsequent follow-up test is performed at the discretion of the investigator to check the status of the laboratory abnormality.

Variations of the embodiments disclosed herein may become apparent to those of ordinary skill in the art. The inventors expect that skilled artisans can employ such variations as appropriate. Accordingly, the invention includes such modifications and equivalents of the subject matter recited in the appended claims.

What is claimed is:

1. A method for treating sialorrhea, comprising the steps of:
identifying a patient afflicted with sialorrhea; and
administering a therapeutically effective amount of glycopyrrolate to the patient using a transdermal route of administration.

2. The method of claim 1, wherein the therapeutically effective amount of glycopyrrolate comprises from about 0.0001 mg/kg/day to about 300 mg/kg/day.

3. The method of claim 2, wherein the therapeutically effective amount of glycopyrrolate comprises from about 0.0005 mg/kg/day to about 50 mg/kg/day.

4. The method of claim 3, wherein the therapeutically effective amount of glycopyrrolate comprises from about 0.001 mg/kg/day to about 10 mg/kg/day.

5. The method of claim 1, wherein the patient afflicted with sialorrhea suffers from a neurological dysfunction.

6. The method of claim 5, wherein the neurological dysfunction comprises Parkinson's Disease, stroke, cerebral palsy, amyotrophic lateral sclerosis, or mental retardation.

7. The method of claim 1, wherein the patient suffers from facial paralysis or cancer about the face, neck or esophagus.

8. The method of claim 1, wherein the transdermal route of administration comprises a single layer drug-in-adhesive patch, a multi-layer drug-in-adhesive patch, a matrix patch, or a reservoir patch.

9. The method of claim 8, wherein the transdermal route of administration comprises at least one adhesive to adhere the patch to the patient.

10. The method of claim 9, wherein the at least one adhesive comprises acrylics, vinyl acetates, natural and synthetic rubbers, ethylene-vinyl acetate copolymers, polysiloxanes, polyacrylates, polyurethanes, plasticized polyether block amide copolymers, plasticized styrene-rubber block copolymers, and mixtures thereof.

11. The method of claim 1, wherein the transdermal route of administration comprises at least one skin penetration enhancer to enhance penetration of glycopyrrolate.

12. The method of claim 11, wherein the at least one skin penetration enhancer comprises fatty acids or salts thereof, fatty alcohols, branched aliphatic alcohols, fatty acid alkyl esters, fatty acid monoesters of sorbitol and glycerol, fatty acid esters with glycolic acid and lactylic acid and salts thereof, fatty acid amides, alkylpyrrolidones, or mixtures thereof.

13. A transdermal drug delivery system for treating a patient exhibiting sialorrhea, comprising:
a transdermal patch;
a therapeutically effective amount of glycopyrrolate contained in said transdermal patch to alleviate sialorrhea; and
a pharmaceutically acceptable carrier;
wherein said transdermal patch comprises a single layer drug-in-adhesive patch, a multi-layer drug-in-adhesive patch, a matrix patch, or a reservoir patch.

14. The transdermal drug delivery system of claim 13, further comprising at least one adhesive about said transdermal patch to adhere said transdermal patch to the patient.

15. The transdermal drug delivery system of claim 14, wherein said at least one adhesive comprises acrylics, vinyl acetates, natural and synthetic rubbers, ethylene-vinyl acetate copolymers, polysiloxanes, polyacrylates, polyurethanes, plasticized polyether block amide copolymers, plasticized styrene-rubber block copolymers, and mixtures thereof.

16. The transdermal drug delivery system of claim 13, wherein said therapeutically effective amount of glycopyrrolate comprises from about 0.0001 mg/kg/day to 300 mg/kg/day.

17. The transdermal drug delivery system of claim 16, wherein said therapeutically effective amount of glycopyrrolate comprises from about 0.0005 mg/kg/day to about 50 mg/kg/day.

18. The transdermal drug delivery system of claim 17, wherein said therapeutically effective amount of glycopyrrolate comprises from about 0.001 mg/kg/day to about 10 mg/kg/day.

19. The transdermal drug delivery system of claim 13, wherein said pharmaceutically acceptable carrier comprises a viscous material suitable for inclusion in said reservoir patch.

20. The transdermal composition of claim 13, wherein said pharmaceutically acceptable carrier comprises a biocompatible polymer.

21. The transdermal drug delivery system of claim 13, further comprising a pharmaceutically acceptable counter ion.

22. The transdermal drug delivery system of claim 21, wherein said pharmaceutically acceptable counter ion comprises chloride, bromide, iodide, acetate, 2-ethylhexanoate, sulfate, phosphate, arylsulfonates, cyclohexylsulfamate, benzoate, saccharinate, or a mixture thereof.

23. The transdermal drug delivery system of claim 13, further comprising at least one skin penetration enhancer to enhance penetration of glycopyrrolate.

24. The transdermal drug delivery system of claim 23, wherein the at least one skin penetration enhancer comprises fatty acids or salts thereof, fatty alcohols, branched aliphatic alcohols, fatty acid alkyl esters, fatty acid monoesters of sorbitol and glycerol, fatty acid esters with glycolic acid and lactylic acid and salts thereof, fatty acid amides, alkylpyrrolidones, or mixtures thereof.

25. The transdermal drug delivery system of claim 23, wherein the at least one skin penetration enhancer comprises oleic acid; lauric acid; oleyl alcohol; lauryl alcohol; 2-butyl-octanol; 2-hexyl decanol; 2-octyl-decanol; 2-hexyldodecanol; 2-octyl-dodecanol; 2-decyl-tetradecanol; 2-tetradecyl-octadecanol; methyl and ethyl laurate; sorbitan monooleate and monolaurate; glycerol monooleate and monolaurate; lauric, myristic, capric, stearic, and oleic diethanolamide; lauric, myristic, capric, stearic, and oleic monoethanolamide; lauric, myristic, capric, stearic, and oleic monoisopropanolamide; caproyl, lauroyl and stearoyl lactylic acid and their salts; caproyl, lauroyl and stearoyl glycolic acid and their salts; N-n-octyl and N-n-dodecyl pyrrolidone.
